(19) United States
(12) Patent Application Publication
(10) Pub. No.: US 2009/0030207 A1
(43) Pub. Date: Jan. 29, 2009

(54) Polymorphs of Dolasetron base and process for preparation thereof

(76) Inventors: Janos Hajko, Debrecen (HU); Tivadar Tamas, Debrecen (HU); Adrienne Kovacsne-Mezei, Debrecen (HU); Piroska Kovacs, Debrecen (HU)

Correspondence Address: KENYON & KENYON LLP, ONE BROADWAY, NEW YORK, NY, 10004, US

(21) Appl. No.: 12/220,084

(22) Filed: Jul. 21, 2008

Related U.S. Application Data
(60) Provisional application No. 60/961,383, filed on Jul. 20, 2007.
(60) Provisional application No. 60/980,661, filed on Oct. 17, 2007.
(60) Provisional application No. 60/986,485, filed on Nov. 8, 2007.
(60) Provisional application No. 61/014,160, filed on Dec. 17, 2007.
(60) Provisional application No. 61/043,878, filed on Apr. 10, 2008.

(51) Int. Cl. C07D 455/00

(52) U.S. Cl. 546/94

(57) ABSTRACT

The present invention provides polymorphic forms of dolasetron base and methods for their use and preparation.

### CROSS REFERENCE TO RELATED APPLICATIONS

The present invention claims the benefit of the following U.S. Provisional Patent Application Nos.: 60/961,383, filed Jul. 20, 2007; 60/980,661, filed Oct. 17, 2007; 60/986,485, filed Nov. 8, 2007; 61/014,160, filed Dec. 17, 2007; and 61/043,878, filed Apr. 10, 2008. The contents of these applications are incorporated herein by reference.

### FIELD OF THE INVENTION

The present invention relates to polymorphic forms of Dolasetron base, and process for preparing said forms.

### BACKGROUND OF THE INVENTION

Dolasetron base is the key intermediate of Dolasetron salts, such as, Dolasetron Mesylate monohydrate. Dolasetron mesylate monohydrate, (2α,6α,8α,9αβ)-octahydro-3-oxo-2,6-methano-2H-quinolizin-8-yl-1H-indole-3-carboxylate monomethanesulfonate monohydrate (referred to as DLS-MsOH.H₂O), a compound having the chemical structure

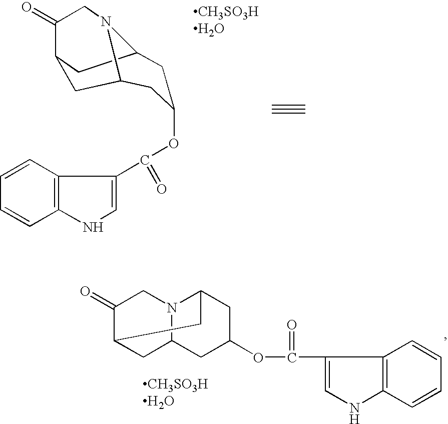

is a serotonin receptor (5-HT₃) antagonist used as an antiemetic and antinauseant agent in chemo- and radiotherapies.

Dolasetron mesylate (DLS-MsOH) developed by Merrell Dow Pharmaceuticals is marketed as tablets for oral administration and as sterile solution for intravenous administration by Aventis, under the name Anzemet®.

EP patent No. 0339669 describes the preparation of Dolasetron-base and its crystallization from a mixture of ethyl acetate and hexane.

WO2007081907 discloses crystalline polymorphic forms A, B, C, D, E, and pure F of Dolasetron base, and processes for preparing said crystalline forms. The crystalline form of Dolasetron base, designated Form C, is characterized by a powder XRD pattern with peaks at about 8.2, 11.7, 13.9±0.2 degrees 2-theta. Form C may be further characterized by a powder XRD pattern with peaks at about 12.8, 15.0, 16.4, 16.8, 17.1, 18.1, 25.3±0.2 degrees 2-theta. Form C may also be substantially identified by the powder XRD pattern as depicted in FIG. 6. The crystalline form of dolasetron base, designated as chemically pure form F, characterized by a powder XRD pattern with peaks at 7.6, 12.1 and 13.7±0.2 degrees 2-theta, and without one or more of the peaks selected from the group consisting of: 11, 14.8, and 22.2±0.2 degrees 2-theta, and combination thereof. Pure form F may be further characterized by a powder XRD pattern with peaks at 11.3, 16.5, 18.2, 19.9, 21.3, 27.8, and 28.5±0.2 degrees 2-theta. Pure form F may also be substantially identified by the powder XRD pattern as depicted in FIG. 7.

WO2007/072507 discloses crystalline polymorphic forms II, III, IV and V of dolasetron base and processes for their preparation.

Further, a publication with reference number IPCOM000144657D reports anhydrous dolasetron base form F and preparation thereof. The crystalline form, designated form F, is characterized by PXRD peaks at 11.0, 11.3, 13.7, and 26.2±0.2 degrees 2-theta. Form F may be further characterized by PXRD peaks at 7.6, 12.1, 14.2, 16.5, 18.2, and 21.6±0.2 degrees 2-theta. Form F may also be substantially identified by the powder XRD pattern as depicted in FIG. 8.

In addition, polymorphs of dolasetron mesylate are described in EP patent No. 0266730, WO2006/026927, WO2007/072506, and in WO2007/081909.

Polymorphism, the occurrence of different crystal forms, is a property of some molecules and molecular complexes. A single molecule, like Dolasetron base, may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubilities.

The discovery of new polymorphic forms of Dolasetron base provides a new opportunity to improve the performance of the synthesis of the active pharmaceutical ingredient (API), Dolasetron mesylate, by producing polymorphs of Dolasetron base having improved characteristics, such as flowability, and solubility. Thus, there is a need in the art for polymorphic forms of Dolasetron base.

### SUMMARY OF THE INVENTION

In one embodiment the present invention encompasses crystalline Dolasetron base characterized by data selected from the group consisting of: a powder XRD pattern with peaks at about 14.3, 14.9, 16.7, 17.3, and 17.7±0.2 degrees 2-theta, a powder XRD pattern as depicted in FIG. 1; and combination thereof.

In another embodiment, the present invention encompasses a process for preparing crystalline dolasetron base characterized by data selected from the group consisting of: a powder XRD pattern with peaks at about 14.3, 14.9, 16.7, 17.3, and 17.7±0.2 degrees 2-theta, a powder XRD pattern as depicted in FIG. 1; and combination thereof comprising combining wet dolasetron base and toluene to obtain a mixture, and removing water from the mixture to obtain a suspension comprising said crystalline form.

In another embodiment, the present invention encompasses amorphous Dolasetron base.

In yet another embodiment, the present invention encompasses a process for preparing amorphous dolasetron base comprising evaporating a solution of dolasetron base in methylene chloride.

In one embodiment the present invention encompasses crystalline Dolasetron base characterized by data selected from the group consisting of: a powder XRD pattern with peaks at about 7.6, 13.4, 13.7, 18.2, and 19.9±0.2 degrees 2-theta, a powder XRD pattern as depicted in FIG. 3; and combination thereof.

In another embodiment the present invention encompasses process for preparing the above crystalline dolasetron base characterized by data selected from the group consisting of: a powder XRD pattern with peaks at about 7.6, 13.4, 13.7, 18.2, and 19.9±0.2 degrees 2-theta, a powder XRD pattern as depicted in FIG. 3; and combination thereof comprising crystallizing dolasetron base from a mixture comprising ethyl acetate as a solvent, and n-heptane as an anti solvent.

In another embodiment the present invention encompasses a process for preparing a mixture of crystalline dolasetron base characterized by the data selected from the group consisting of: a powder XRD pattern with peaks at about 7.6, 13.4, 13.7, 18.2, and 19.9±0.2 degrees 2-theta, and a powder XRD pattern as depicted in FIG. 3, and crystalline dolasetron base Form C characterized by a powder XRD pattern with peaks at about 8.2, 11.7, 13.9±0.2 degrees 2-theta, comprising: drying dolasetron base at a pressure of less then 500 mbar at a temperature of about at 40° C. to about 120° C.

In another embodiment, the present invention provides a process for preparing Dolasetron salt from anyone of the above forms of dolasetron base and mixtures thereof.

In yet another embodiment, the present invention provides a process for preparing dolasetron salt by preparing any of the above forms of dolasetron base according to the processes of the present invention, and converting them to dolasetron salt.

### BRIEF DESCRIPTION OF THE FIGURES

FIG. 1 shows a powder XRD pattern of crystalline Dolasetron base designated form G.

FIG. 2 shows a powder XRD pattern of amorphous Dolasetron base.

FIG. 3 shows a powder XRD pattern of crystalline Dolasetron base designated form J.

FIG. 4 shows a powder XRD pattern of form G after storing at 80% RH for 1 week.

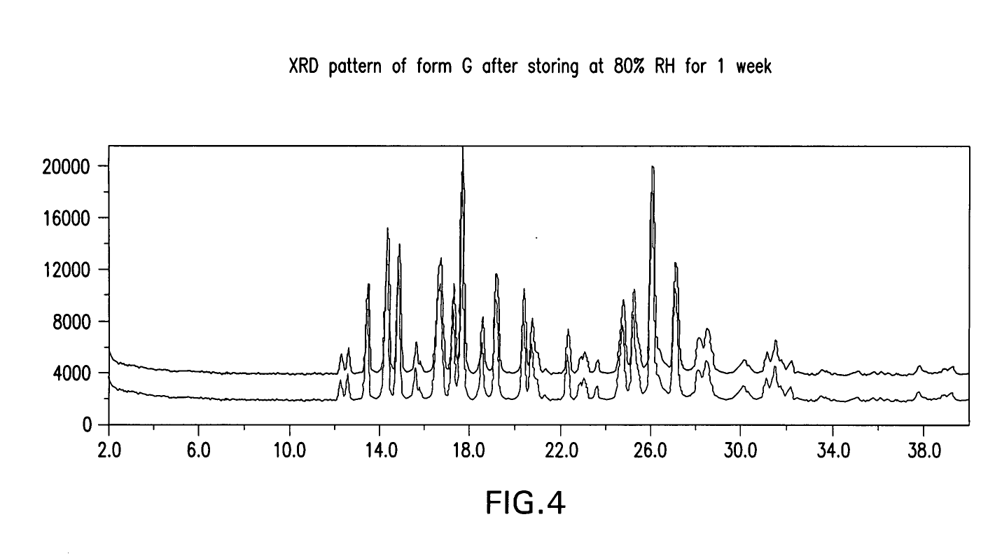

FIG. 5 shows a powder XRD pattern of form J after storing at 80% RH for 1 week.

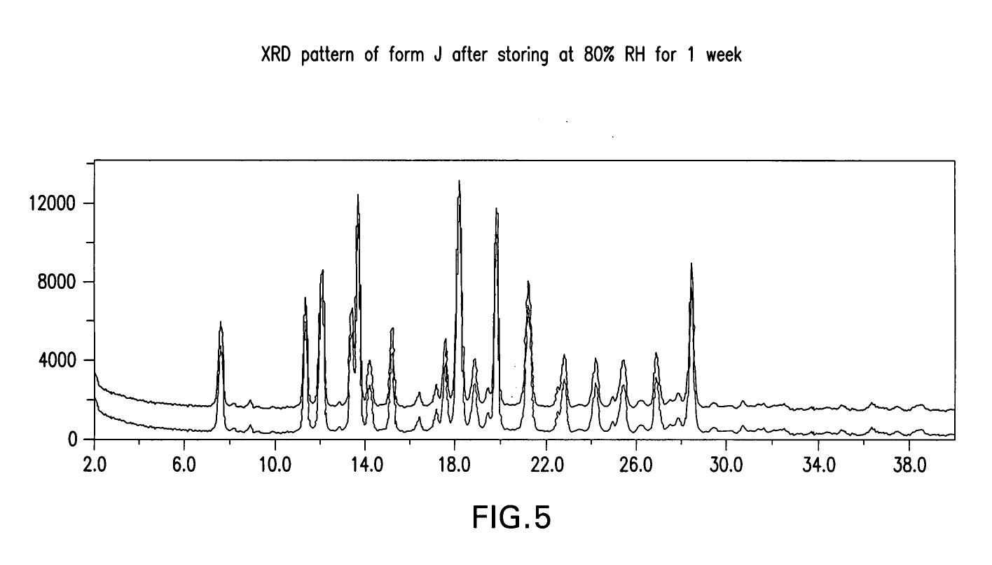

FIG. 6 shows a powder XRD pattern of crystalline Dolasetron base designated form C.

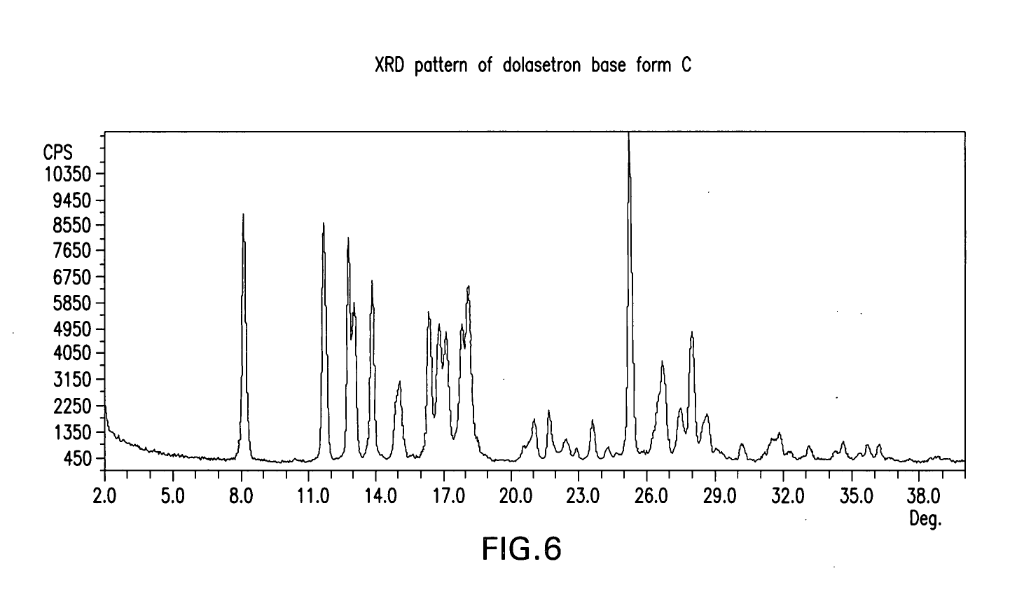

FIG. 7 shows a powder XRD pattern of crystalline Dolasetron base designated chemically pure form F.

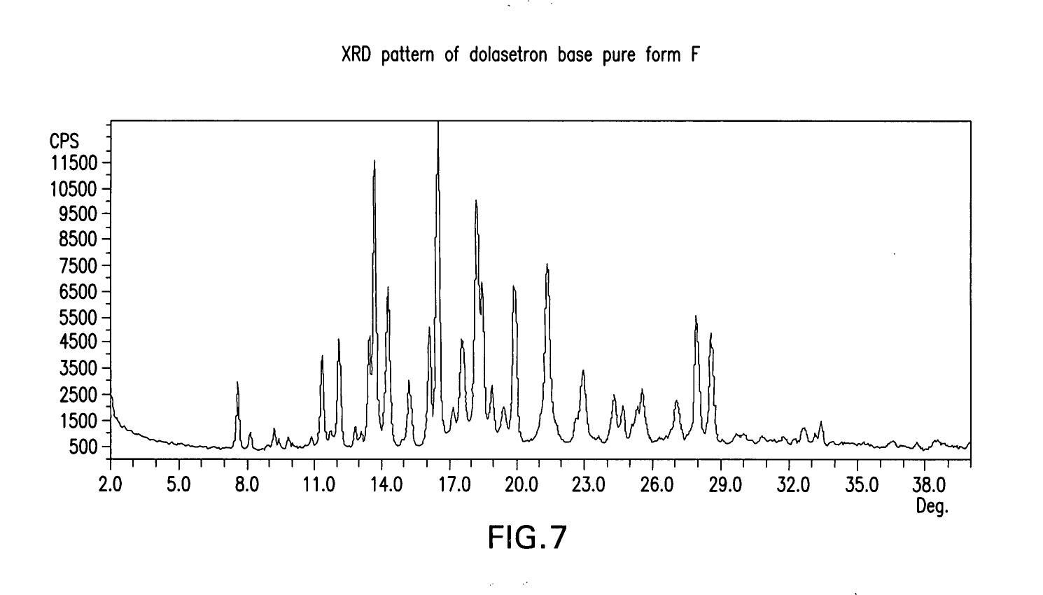

FIG. 8 shows a powder XRD pattern of crystalline Dolasetron base designated form F.

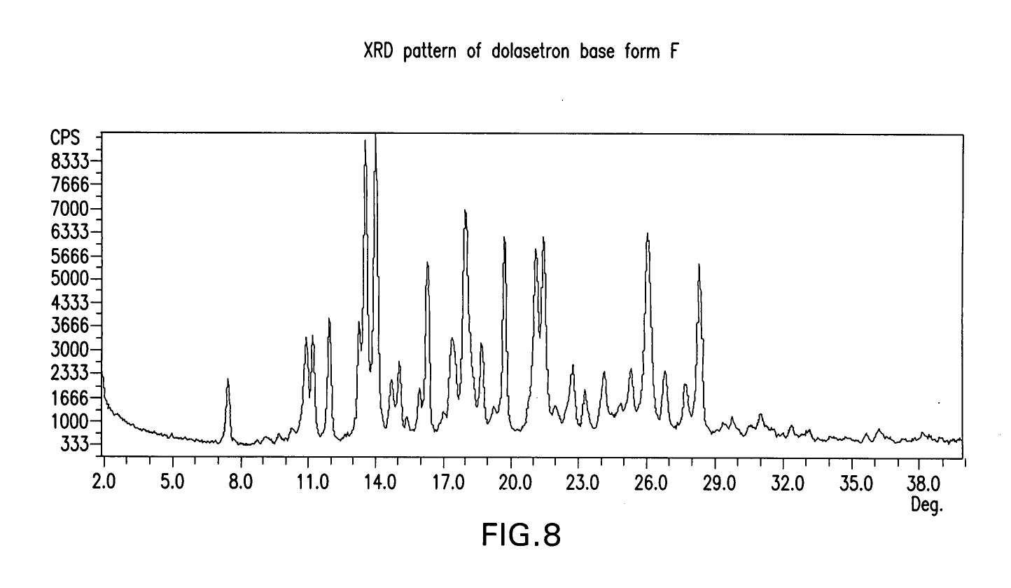

FIG. 9 shows a TGA curve of Dolasetron base form J.

FIG. 10 shows a TGA curve of Dolasetron base form G.

FIG. 11 shows a DSC curve of Dolasetron base form J.

FIG. 12 shows a DSC curve of Dolasetron base form G.

### DETAILED DESCRIPTION OF THE INVENTION

The present invention relates to polymorphic forms of Dolasetron base, and process for preparation thereof.

As used herein crystalline dolasetron form A refers to crystalline dolasetron characterized by a powder XRD pattern with peaks at about 12.9, 14.1, and 15.2.±0.2 degrees 2-theta, crystalline dolasetron form B refers to crystalline dolasetron characterized by a powder XRD pattern with peaks at about 13.6, 16.4, and 23.4.±0.2 degrees 2-theta, crystalline dolasetron form D refers to crystalline dolasetron characterized by a powder XRD pattern with peaks at about 14.3, 16.1, 16.5 and 18.5±0.2 degrees 2-theta.

As referenced herein peak positions from an X ray diffraction pattern have a variation of ±0.2 degrees 2-theta. The accuracy of peak positions is defined as ±0.2 degrees 2-theta due to experimental differences such as instrumentation, sample preparations etc.

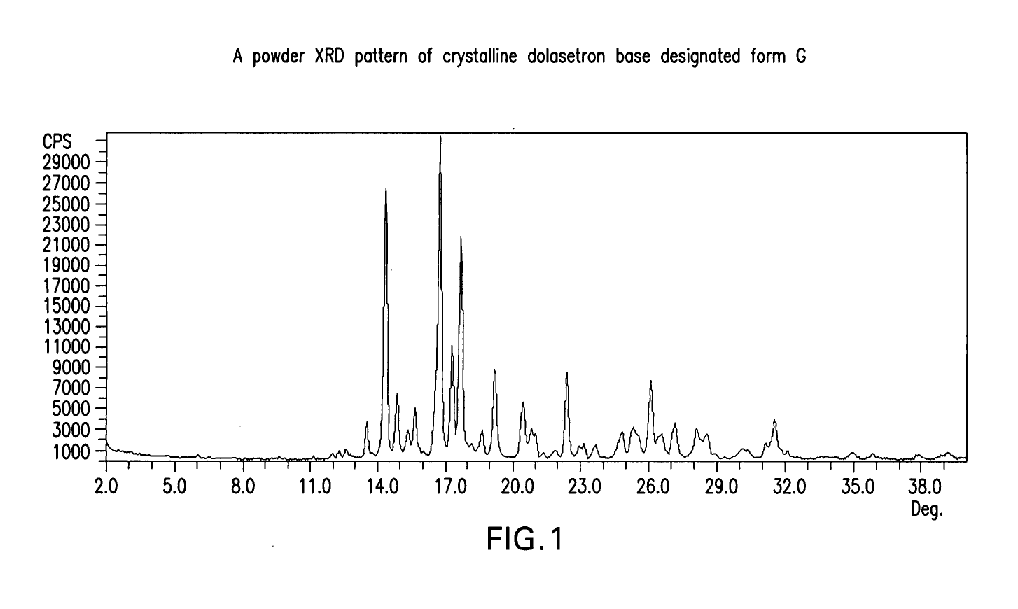

The present invention encomposes crystalline Dolasetron base, designated form G, characterized by data selected from the group consisting of: a powder XRD pattern with peaks at about 14.3, 14.9, 16.7, 17.3, and 17.7±0.2 degrees 2-theta, a powder XRD pattern as depicted in FIG. 1; and combination thereof.

The above crystalline dolasetron base form G of dolasetron base can be further characterized by a powder XRD pattern with peaks at about 13.5, 15.6, 19.2, 20.4, 22.4, and 26.1±0.2 degrees 2-theta.

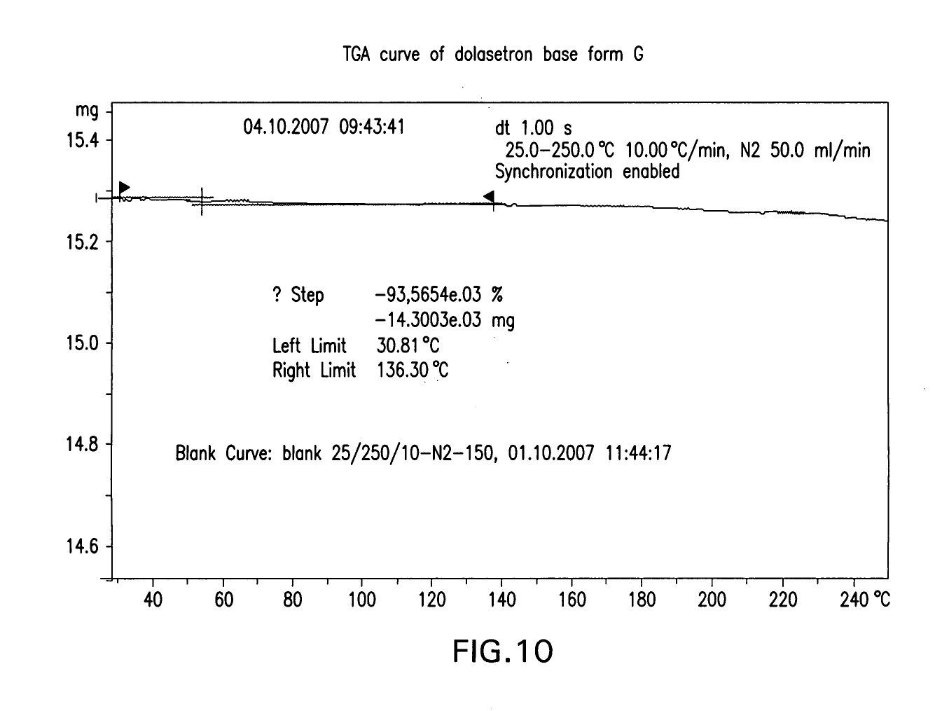

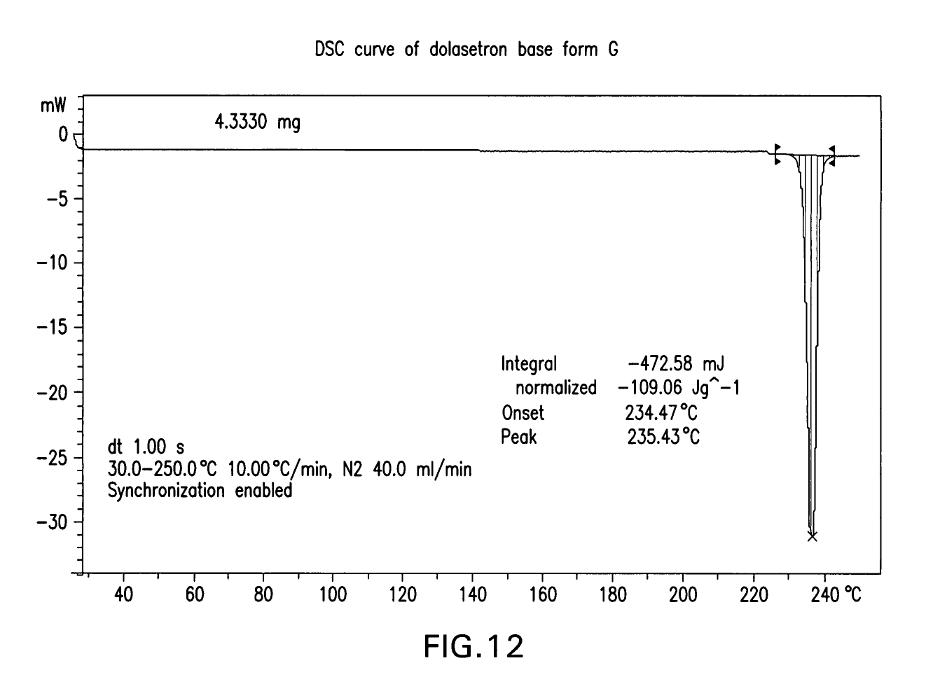

The above crystalline form G of dolasetron base may be further characterized by a weight loss of less than 0.1%, at temperatures of about 160° C., as measured by TGA. The above crystalline form G of dolaseton base may be further characterized by a TGA thermogram as depicted in FIG. 10. The above crystalline form may be further characterized by a DSC thermogram having a sharp endothermic peak at about 235-237° C. Crystalline form G of dolasetron base may be further characterized by a DSC thermogram as depicted in FIG. 12.

Crystalline form G of dolasetron base is an anhydrous form of Dolasetron base. As used herein, the term “anhydrous” in reference to Crystalline form G of dolasetron base, refers to Crystalline form G of dolasetron base that contains no more than 1% by weight of water or of any solvent as measured by TGA.

The above crystalline Dolasetron base form G has less than 10% by weight, more preferably less than 5% by weight, and most preferably less than 1% by weight of crystalline Dolasetron base, designated pure form F having an X-ray powder diffraction pattern with peaks at about 13.7, 16.1 and 16.5±0.2 degrees 2-theta.

The content of the crystalline Dolasetron base pure form F may be measured by PXRD, using the peak at about 13.7±0.2 degrees 2-theta.

The above crystalline dolasetron base form G is a stable polymorphic form of dolasetron base. As used herein, the term “stable” in reference to dolasetron base form G, refers to dolasetron base form G which does not convert to another polymorph upon storage for at least about one week at relative humidity of no more than about 80% at a temperature of about 22° C. to about 27° C., i.e. having crystalline stability under these conditions.

The above crystalline dolasetron base form G may be prepared by a process comprising providing a solution of dolasetron base in isobutyl acetate, and precipitating the said crystalline dolasetron base at a temperature of about 0° C. to about 10° C.

The solution of dolasetron base in isobutyl acetate is provided by combining dolasetron base and isobutyl acetate to obtain a mixture, and heating the mixture to obtain a solution. Preferably, the mixture is heated to a temperature of about 50° C. to about 120° C., more preferably, to a temperature of about 70° C. to about 100° C., most preferably, to a temperature of about 90° C. to about 100° C.

Optionally, the solution is treated with charcoal, prior to precipitation of the crystalline form. After treating the solution with charcoal, it is filtered.

Preferably, precipitation is carried out by a process comprising concentrating the solution; and cooling the concentrated solution to a temperature of about 0° C. to about 10° C. to obtain a suspension comprising the crystalline dolasetron base form G. Preferably, precipitation is carried out at a temperature of about 2° C. to about 8° C. Preferably, the solution is concentrated at a temperature of about 40° C. to about 60° C., more preferably, at a temperature of about 50° C. to about 60° C.

Preferably, cooling is carried out for a period of about 2 hours to about 24 hours, more preferably, for a period of about 4 hours to about 12 hours.

The process for preparing crystalline dolasetron base form G may further comprise recovering the crystalline dolasetron base from the suspension. The recovery may be carried out for example, by filtering the suspension, washing the filtered precipitate of the crystalline form and drying.

Preferably, drying is at a temperature of about 40° C. to about 120° C., more preferably at a temperature of about 60° C. to about 90° C. Preferably, drying is carried out for a period of about 2 hours to about 24 hours, more preferably for a period of about 4 hours to about 18 hours.

The above crystalline dolasetron base form G can also be prepared by a process comprising combining wet dolasetron base and toluene to obtain a mixture, and removing water from the mixture to obtain a suspension comprising said crystalline form. Preferably, the starting material dolasetron base can be form A, B, D, or mixtures thereof.

As used herein, the term “wet” in reference to dolasetron base, refers to dolasetron base obtained from a recovery process which does not include drying. Typically, such a product contains solvent or a mixture of solvents in an amount of about 0.1% to about 60% by weight as determined by Loss on Drying (“LOD”), wherein the solvent or the mixture of solvents can be an organic solvent, water, and a mixture thereof Preferably, the product contains solvent or a mixture of solvents in an amount of about 0.1% to about 5% by weight as determined by Loss on Drying. (“LOD”). Preferably, form A contains a water content of about 0.1% to about 3% by weight as determined by Loss on Drying. Preferably, forms B and D contain a water content of about 5% by weight determined by Loss on Drying.

As used herein, the drying process includes drying at all temperatures and pressures including room temperature and a pressure of 1 atmosphere.

Preferably, removing water is carried out by heating the mixture of wet dolasetron base and toluene. Preferably, heating is to a temperature of about 100° C. to about 120° C., more preferably, at a temperature of about 100° C. to about 110° C., most preferably, at a temperature of about 105° C. to about 110° C. Preferably, heating is carried out for a period of about 0.5 hours to about 6 hours, more preferably, for a period of about 0.5 hour. Preferably, the heating is carried out using a water trap for azeotropic removal of water.

Preferably, the suspension can be cooled to a temperature of about 30° C. to about 0° C., more preferably, to a temperature of about 25° C. to about 15° C., to increase the yield of the said crystalline dolasetron base. Preferably, cooling is carried out for a period of about 1 hour to about 24 hours, more preferably, for about 2 hours to about 6 hours.

The process for preparing crystalline dolasetron base form G may further comprise recovering the crystalline dolasetron base from the suspension. The recovery may be carried out for example by filtering the suspension, and drying. Preferably, drying is at a temperature of about 40° C. to about 120° C. Preferably, drying is carried out for a period of about 2 hours to about 24 hours.

The present invention provides amorphous Dolasetron base.

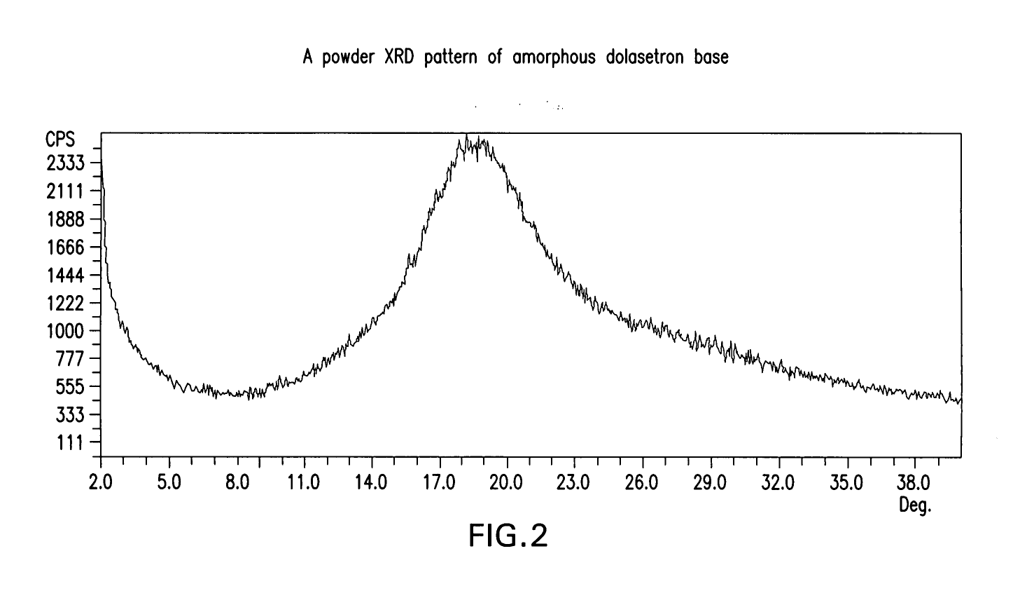

The amorphous dolasetron base can be characterized by a powder XRD pattern as depicted in FIG. 2.

The above amorphous Dolasetron base has less than 10% by weight, more preferably less than 5% by weight, and most preferably less than 1% by weight of crystalline Dolasetron base Form F.

The content of the crystalline Dolasetron base Form F may be measured by PXRD, using the peak at about 16.5±0.2 degrees 2-theta.

The above amorphous dolasetron base can be prepared by a process comprising evaporating a solution of dolasetron base in methylene chloride.

The solution of dolasetron base in methylene chloride may be provided by a process comprising combining dolasetron base and methylene chloride.

The obtained solution is evaporated. The evaporation can be done step wise, i.e., first, the solvent is evaporated at a temperature of about 20° C. to about 40° C., to obtain a residue, which is then further dried at about 40° C. to about 120° C. under vacuo. Preferably, the first evaporation step is done at about 30° C. to about 35° C. Preferably, evaporation is done for a period of about 0.5 hour to about 4 hours. Preferably, evaporation is done under vacuo. Preferably, under vacuo is at a pressure of about 20 mbar to about 500 mbar, most preferably, at about 50 mbar to about 100 mbar. Preferably, the drying is performed for a period of about 2 hours to about 24 hours.

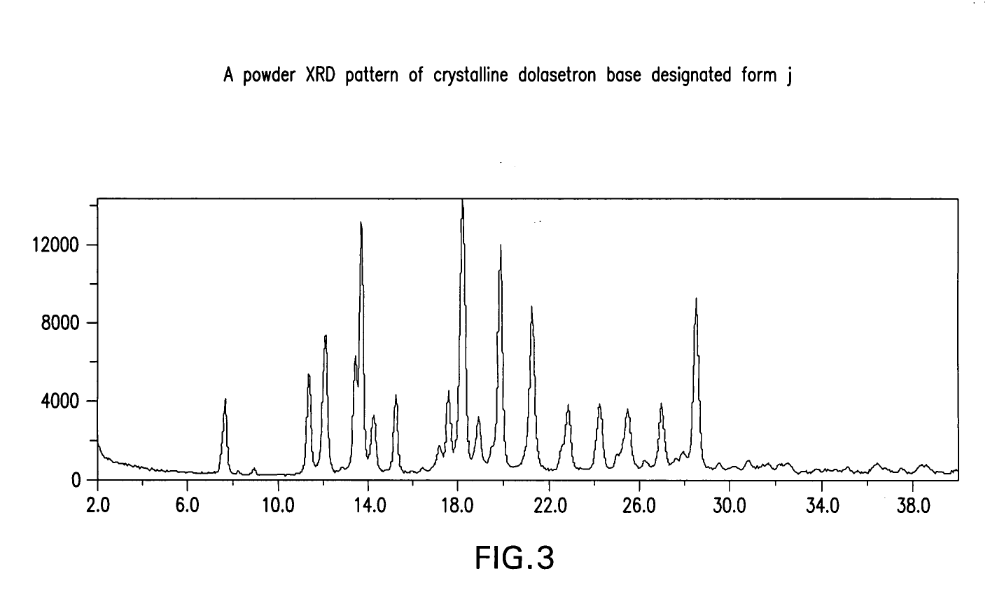

The present invention encompasses crystalline Dolasetron base, designated form J, characterized by data selected from the group consisting of: a powder XRD pattern with peaks at about 7.6, 13.4, 13.7, 18.2, and 19.9±0.2 degrees 2-theta, a powder XRD pattern as depicted in FIG. 3; and combination thereof, having less than 10% by weight, preferably less than 5% by weight more preferably less than 1% by weight of crystalline dolasetron base Form C characterized by a PXRD pattern with peaks at about 8.2, 11.7, 13.9±0.2 degrees 2-theta. The content of the crystalline dolasetron base form C is measured by PXRD using the peak at 8.2±0.2 degrees 2-theta.

The above crystalline dolasetron base form J can be further characterized by a powder XRD pattern with peaks at about 11.3, 12.0, 15.2, 21.2, and 28.4±0.2 degrees 2-theta.

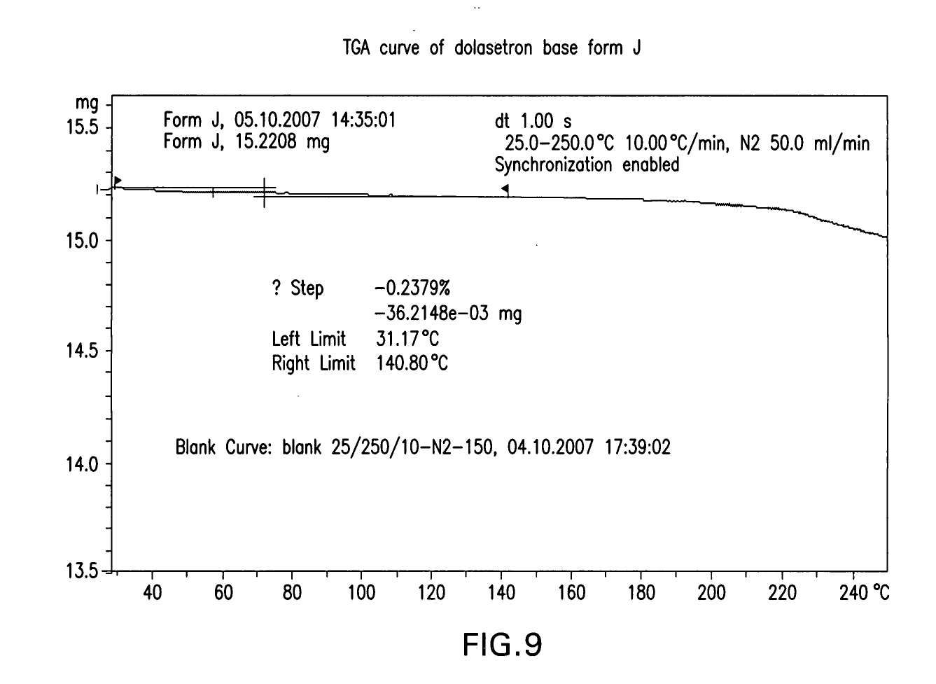

The above crystalline form J may be further characterized by a weight loss of about 0.3% at temperatures of about 140° C. as measured by TGA. The above crystalline form J of dolaseton base may be further characterized by a TGA thermogram as depicted in FIG. 9.

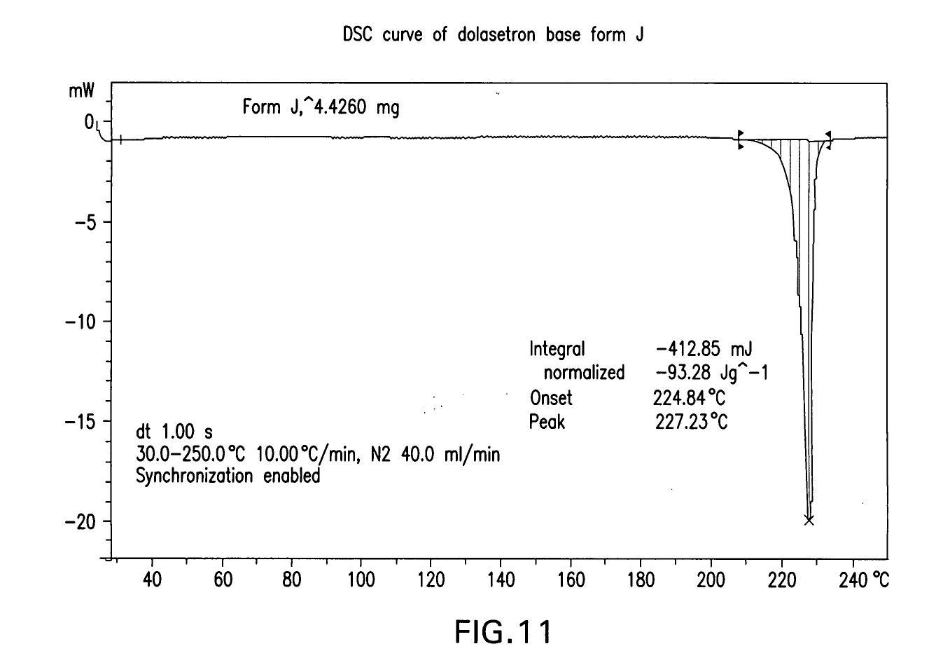

The above crystalline form may be further characterized by a DSC thermogram having a sharp endothermic peak at about 227-228° C. Crystalline form J of dolasetron base may be further characterized by a DSC thermogram as depicted in FIG. 11. The above crystalline form J is an anhydrous form of Dolasetron base.

The above crystalline Dolasetron base form J has less than 10% by weight, more preferably less than 5% by weight, and most preferably less than 1% by weight of crystalline Dolasetron base pure form F. The content of the crystalline Dolasetron base pure form F is measured by PXRD, using the peak at 16.5±0.2 degrees 2-theta

The above crystalline dolasetron base form J is a stable polymorphic form of dolasetron base. As used herein, the term “stable” in reference to dolasetron base form J, refers to dolasetron base form J which does not convert to another polymorph upon storage for about one week at relative humidity of no more than about 80% at a temperature of about 22° C. to about 27° C., i.e. having crystalline stability under these conditions.

In another embodiment the present invention encompasses a process for preparing the above crystalline dolasetron base form J comprising crystallizing dolasetron base from a mixture comprising ethyl acetate, as a solvent, and n-heptane, as an anti solvent.

In the above process crystallization comprises providing a solution of dolasetron base in ethyl acetate, and combining the solution with n-heptane to obtain a suspension.

Preferably, said solution is provided by combining dolasetron base and ethyl acetate to obtain a mixture and heating the mixture to obtain a solution. Preferably the heating is to a temperature of about 40° C. to about 80° C., more preferably, to about 50° C. to about 70° C.

Further, the solution can be added to n-heptane or n-heptane can be added to the solution to obtain the suspension. Preferably, n-heptane is added to the solution.

To increase the yield of the precipitated crystalline dolasetron base, precipitation can be followed by cooling said suspension to a temperature of about 25° C. to about 0° C., preferably, to about 8° C. to about 2° C. Preferably, cooling is carried out for a period of about 2 hours to about 24 hours, more preferably, for about 2 hours to about 6 hours.

The process for preparing the said crystalline dolasetron base form J may further comprise recovering the crystalline dolasetron base from the suspension. The recovery may be done for example, by filtering the suspension, and drying. Preferably, drying is carried out at a temperature of about 40° C. to about 120° C., more preferably of 50° C. to about 100° C., most preferably of 75° C. to about 85° C. Preferably, the drying is performed overnight.

In another embodiment the present invention provides a process for preparing a mixture of crystalline dolasetron base, designated form J, and crystalline dolasetron base Form C characterized by a powder XRD pattern with peaks at about 8.2, 11.7, 13.9±0.2 degrees 2-theta, comprising: drying dolasetron base forms B and D at a pressure of less then 500 mbar at a temperature of about 40° C. to about 120° C. Preferably, drying is carried out at a pressure of less then 200 mbar more preferably, at about 30 mbar to about 100 mbar. Preferably, drying is carried out at about 70° C. to about 120° C., more preferably, 80° C. to about 100° C.

Preferably, drying is carried out for a period of about 1 hour to about 24 hours, more preferably 2 hours to about 16 hours, most preferably 3 hours to about 10 hours.

The starting dolasetron base used to prepare forms G, J and a mixture of C+J can be prepared, for example according to a process disclosed in US 2007/0203176.

The forms of dolasetron base disclosed herein and combination thereof, can be used to prepare a dolasetron salt, preferably dolasetron mesylate.

The present invention encomposes a process for preparing a dolasetron salt, comprising preparing any one of the above polymorphs of Dolasetron base or mixtures thereof and converting them to dolasetron salt. Preferably, the polymorphs of dolasetron base are prepared by the process disclosed herein.

The conversion of dolasetron base to a dolasetron salt may be carried out by reacting dolasetron base with an acid. Preferably, the acid is methanesulfonic acid. Preferably, the Dolasetron salt is Dolasetron mesylate monohydrate. Where a methanesulfonic acid salt form of dolasetron is prepared the reaction may comprise, combining dolasetron base, methanesulfonic acid, and a solvent mixture comprising acetone and water to obtain a suspension. Preferably, methanesulfonic acid is added to a suspension of the dolasetron base in a mixture comprising acetone and water. Preferably, the ratio of acetone and water is about 99:1 to about 80:20, preferably about 95:5.

Preferably, the addition of methane sulfonic acid transforms the suspension into a solution, in which a precipitate is formed after a few minutes.

The suspension is cooled to increase the yield of the precipitated dolasetron mesylate monohydrate. Preferably, the suspension is cooled to a temperature of about 0° C. to about 10° C., more preferably to about 2° C. to about 8° C. Preferably, cooling is carried out for a period of about an hour to about 24 hours, more preferably for about 2 to about 8 hours, most preferably for about 4 hours.

The process may further comprise recovering the Dolasetron mesylate. The recovery may be carried out by any method known to a skilled artisan. Preferably, the recovery may be carried out for example by filtration of the cooled suspension, washing the filtered product, and drying it.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to limit its scope in any way.

### EXAMPLES

### Differential Scanning Calorimetry (DSC)

DSC 822^{e}/700, Mettler Toledo, Sample weight: 3-5 mg.
Heating rate: 10° C./min., Number of holes of the crucible: 3
In N₂ stream: flow rate=40 ml/min
Scan range: 30-250° C., 10° C./ minutes heating rate.

### Thermal Gravimetric Analysis (TGA)

TGA/SDTA 851^{e}, Mettler Toledo , Sample weight 7-15 mg.
Heating rate: 10° C./min., In N₂ stream: flow rate=50 ml/min
Scan range: 30-250° C.

### PXRD

ARL X-ray powder diffractometer model X'TRA-030, Peltier detector, round standard aluminum sample holder with round zero background quartz plate was used. Scanning parameters: Range: 2-40 deg. 2θ, continuous Scan, Rate: 3 deg./min. The accuracy of peak positions is defined as +/−0.2 degrees due to experimental differences like instrumentations, sample preparations etc.

### Example 1

### Preparation of Crystalline Dolasetron Base Designated Form G

Crude Dolasetron base (1 kg) was dissolved in isobutyl acetate (40 L) at 70-100° C. Charcoal (0.1 kg) was added to the solution, and after 30 minutes of stirring it was filtered off, and washed with isobutyl acetate (4 L). The solution was evaporated under reduced pressure at 40-60° C. to obtain a residue (ca 6 L), which was cooled to 0-10° C., and stirred at this temperature for min. 6 hours. The precipitated crystals were filtered off, washed with isobutyl acetate (1 L), and dried overnight at 37-43° C. under reduced pressure. Polymorphism was determined by X-ray diffraction.

### Example 2

### Preparation of Crystalline Dolasetron Base Designated Form G

A mixture of Dolasetron bases forms B and D having a water content of up to 5% (2 g) was heated in toluene (60 ml) under reflux (cooler was equipped with water-trap) for 1 hour to obtain a suspension. The suspension was cooled to 15-25° C. and allowed to crystallize at this temperature overnight. The crystals were filtered off, dried in vacuum at 80° C. for 6 hours. Polymorphism was determined by XRD pattern as depicted in FIG. 1.

### Example 3

### Preparation of Crystalline Dolasetron Base Designated Form G

Dolasetron base form A (2 g) was heated in toluene (60 ml) under reflux (cooler was equipped with water-trap) for 30 minutes. The suspension was cooled to 15-25° C. and allowed to crystallize at this temperature overnight. The crystals were filtered off, and dried in vacuum at 80° C. for 6 hours. Polymorphism was determined by XRD pattern as depicted in FIG. 1.

### Example 4

### Preparation of Amorphous Dolasetron Base Characterized by a Powder XRD Pattern as Depicted in FIG. 2

A mixture of Dolasetron bases B and D was dissolved in methylene chloride. The resulting solution was evaporated completely at a temperature of about 30° C. to about 35° C. to obtain a residue which was dried overnight at 60° C. under reduced pressure, less then 200 mbar. Polymorphism was determined by a powder XRD pattern as depicted in FIG. 2.

### Example 5

### Preparation of Crystalline Dolasetron Base Designated Form J

A mixture of Dolasetron bases B and D (2 g) was dissolved in ethyl acetate (80 ml) at 60° C. n-Heptane (160 ml) was added to the solution and the suspension was cooled to 2-8° C. and allowed to crystallize at this temperature overnight. The crystals were filtered off, dried in vacuum at 80° C. overnight. Polymorphism was determined by X-ray diffraction as depicted in FIG. 3.

### Example 6

### Preparation of a Mixture of Crystalline Dolasetron Base Forms J and C

A mixture of Dolasetron bases B and D was dried in vacuum at 80° C. under nitrogen for 6 hours. Polymorphism was determined by X-ray diffraction. Dry sample was a mixture of form C and J.

### Example 7

### Preparation of DLS-MsOH Monohydrate

Methanesulfonic acid (2.85 ml, 1 equiv) was added to a stirred suspension of Dolasetron base G(14.24 g, 43.9 mmol) in a mixture of acetone-water 95:5 (100 ml). The solid dissolved immediately, after some minutes the salt precipitated in crystalline form. The mixture was put into fridge, after 4 hours the salt was filtered off, washed with same solvent mixture (2±15 ml), and dried overnight in an air-ventilated oven at 40° C. The yield was 15.63 g (81%).

### Example 8

### Hygroscopicity Table and Stability Measurements of Form G and J

| INITIAL FORM: Form J | | | INITIAL FORM: Form G | | |
|---|---|---|---|---|---|
| INITIAL water content: <0.1% | | | INITIAL water content: 0.1% | | |
| Duration: 1 week | | | Duration: 1 week | | |
| Temperature: 25 ± 2° C. | | | Temperature: 25 ± 2° C. | | |
| % Relative | | CRYSTAL | % Relative | | CRYSTAL |
| Humidity | WATER CONTENT | FORM | Humidity | WATER CONTENT | FORM |
| 40 | <0.1% | J | 40 | <0.1% | G |
| 60 | <0.1% | J | 60 | <0.1% | G |
| 80 | 0.2% | J | 80 | <0.1% | G |
| 100 | 5.5% | B >> J | 100 | 4.1% | B >> G |

### Example 9

### Preparation of Starting Dolasetron Base (According to US 2007/0203176)

Indole-3-carboxylic acid (17.7 g, 1.1 equiv.) was added in portions to a solution of trifluoroacetic anhydride (20 ml, 1.4 equiv.) in a mixture toluene (360 ml) and trifluoroacetic acid (90 ml), at room temperature (20-25° C.), during 15 minutes. After 5-minutes of stirring, endo-5-hydroxy-8-azatricyclo[5.3.1.0^{3,8}]-undecan-10-one (18.12 g, 0.1 mol), was added in one portion. The reaction mixture heated to 30-35° C., the solid phase dissolved. The solution was stirred for 2 hours without external heating. The trifluoroacetic acid was removed by evaporation under reduced pressure until starting of crystallization. 10% of an aqueous solution of sodium carbonate (360 ml) was added, then toluene was removed by evaporation under reduced pressure. The precipitated Dolasetron base monohydrate was collected by filtration, washed with water (3×60 ml), and dried overnight at 40° C. under reduced pressure. The dry product was weighed as 33.63 g (98%).

What is claimed is:

1. Crystalline dolasetron base characterized by data selected from the group consisting of: a powder XRD pattern with peaks at about 14.3, 14.9, 16.7, 17.3, and 17.7±0.2 degrees 2-theta, a powder XRD pattern as depicted in FIG. 1; and combination thereof.

2. Crystalline dolasetron base of claim 1, characterized by a powder XRD pattern with peaks at about 14.3, 14.9, 16.7, 17.3, and 17.7±0.2 degrees 2-theta.

3. Crystalline dolasetron base of claim 1, characterized by a powder XRD pattern as depicted in FIG. 1.

4. Crystalline dolasetron base of claim 2, further characterized by a powder XRD pattern with peaks at about 13.5, 15.6, 19.2, 20.4, 22.4, and 26.1±0.2 degrees 2-theta.

5. Crystalline dolasetron base of claim 1, further characterized by a weight loss of less than 0.1%, at temperatures of about 160° C., as measured by TGA.

6. Crystalline dolasetron base of claim 1, further characterized by a DSC thermogram having a sharp endothermic peak at about 235-237° C.

7. Crystalline dolasetron base of claim 1, having less than 10% by weight of a crystalline Dolasetron base characterized by a powder XRD pattern with peaks at about 13.7, 16.1 and 16.5±0.2 degrees 2-theta.

8. Crystalline dolasetron base of claim 1, wherein the dolasetron base is anhydrous.

9. Crystalline dolasetron base of claim 1, wherein the crystalline form is a stable polymorphic form upon storage for at least about a week at a relative humidity of no more than about 80% at a temperature of about 22° C. to about 27° C.

10. A method for preparing a crystalline form of dolasetron base characterized by data selected from the group consisting of: a powder XRD pattern with peaks at about 14.3, 14.9, 16.7, 17.3, and 17.7±0.2 degrees 2-theta, a powder XRD pattern as depicted in FIG. 1; and combination thereof, by a process comprising combining wet dolasetron base and toluene to obtain a mixture and removing water from the mixture to obtain a suspension comprising the crystalline form.

11. The process of claim 10, wherein removing water is carried out by heating the mixture of wet dolasetron base and toluene to a temperature of about 100° C. to about 120° C.

12. The process of claim 11, wherein heating is carried out with a water trap for azeotropic removal of water.

13. The process of claim 10, wherein the suspension is cooled to a temperature of about 30° C. to about 0° C.

14. The process of claim 10, further comprising recovering the crystalline dolasetron base from the suspension.

15. Dolasetron base selected from the group consisting of amorphous dolasetron base; and crystalline dolasetron base characterized by data selected from the group consisting of: a powder XRD pattern with peaks at about 7.6, 13.4, 13.7, 18.2, and 19.9±0.2 degrees 2-theta, a powder XRD pattern as depicted in FIG. 3, and combination thereof, having less than 10% by weight of crystalline dolasetron base Form C characterized by a PXRD pattern with peaks at about 8.2, 11.7, 13.9±0.2 degrees 2-theta.

16. Crystalline dolasetron base of claim 15, characterized by a powder XRD pattern with peaks at about 7.6, 13.4, 13.7, 18.2, and 19.9±0.2 degrees 2-theta.

17. Crystalline dolasetron base of claim 15, characterized by a powder XRD pattern as depicted in FIG. 3.

18. Crystalline dolasetron base of claim 16, further characterized by a powder XRD pattern with peaks at about 11.3, 12.0, 15.2, 21.2, and 28.4±0.2 degrees 2-theta.

19. Crystalline dolasetron base of claim 15, characterized by a weight loss of about 0.3% at temperatures up to about 140° C. as measured by TGA.

20. Crystalline dolasetron base of claim 15, characterized by a DSC thermogram having a sharp endothermic peak at about 227-228° C.

21. Crystalline dolasetron base of claim 15, characterized by having less than 10% by weight of crystalline Dolasetron base characterized by a powder XRD pattern with peaks at about 13.7, 16.1 and 16.5±0.2 degrees 2-theta.

22. Crystalline Dolasetron base of claim 15, wherein the dolasetron base is an anhydrous form.

23. Crystalline dolasetron base of claim 15, wherein crystalline form is a stable polymorphic form upon storage for at least about a week at a relative humidity of no more than about 80% at a temperature of about 22° C. to about 27° C.

24. Amorphous dolasetron base of claim 15.

25. Amorphous dolasetron base of claim 15, characterized by a powder XRD pattern as depicted in FIG. 2.

26. Amorphous dolasetron base of claim 15, characterized by having less than about 10% by weight of a crystalline dolasetron base characterized by a powder XRD pattern with peaks at about 13.7, 16.1 and 16.5±0.2 degrees 2-theta.

27. A process for preparing a dolasetron salt comprising providing a dolasetron base according to claim 1; and converting said dolasetron base to a dolasetron salt.

28. A process for preparing a dolasetron salt, comprising
a) preparing a dolasetron base according to claim 10; and
b) converting said dolasetron base form prepared in step a) to said dolsetron salt.

29. The process of claim 27 wherein the dolasetron salt is a dolasetron mesylate salt.

30. A process for preparing a dolasetron salt comprising providing a dolasetron base according to claim 15; and converting said dolasetron base to a dolasetron salt.

31. The process of claim 30, wherein the dolasetron salt is a dolasetron mesylate salt.
